(19) United States
(12) Patent Application Publication
(10) Pub. No.: US 2009/0157022 A1
(43) Pub. Date: Jun. 18, 2009

(54) ABSORBENT ARTICLES HAVING A WETNESS INDICATOR

(75) Inventors: John Gavin MacDonald, Decatur, GA (US); Terry Lee Combs, Alpharetta, GA (US); Andrew Mark Long, Appleton, WI (US); Mary L. McDaniel, Appleton, WI (US); Jaeho Kim, Roswell, GA (US)

Correspondence Address: DORITY & MANNING, P.A., POST OFFICE BOX 1449, GREENVILLE, SC, 29602-1449, US

(73) Assignee: KIMBERLY-CLARK WORLDWIDE, INC., Neenah, WI (US)

(21) Appl. No.: 11/955,916

(22) Filed: Dec. 13, 2007

(51) Int. Cl. A61F 13/15

(52) U.S. Cl. 604/361; 604/379; 604/385.23

(57) ABSTRACT

Absorbent articles including a wetness indicator are generally described. The wetness indicator of the present invention is configured to expand toward the skin of the wearer (i.e., in the z-direction of the absorbent article perpendicular to the plane of the absorbent article) upon contact with a liquid. However, the expansion of the wetness indicator is substantially limited to the z-direction. That is, the wetness indicator does not substantially expand in any direction parallel with the plane of the article (i.e., the x- and y-directions). As such, the wetness indicator does not significantly interfere with the absorbent capabilities of the absorbent article. Thus, the wetness indicator can be included within conventional absorbent articles without significantly sacrificing the absorbency characteristics of the article.

### BACKGROUND OF THE INVENTION

Many articles intended for personal wear (e.g., such as diapers, training pants, feminine hygiene products, adult incontinence products, bandages, medical garments and the like) are designed absorb moisture from liquid body exudates including urine, menses, blood, etc. and pull moisture away from the wearer to reduce skin irritation caused by prolonged wetness exposure. In some instances, it may be desirable to give a signal (e.g., an uncomfortable and/or wet feeling against the skin) to alert the wearer that the act of urination has occurred. On the other hand, there is a counter-balancing concern about the possibility of skin irritations and rashes caused by prolonged wetness against the skin if the articles are less absorbent to allow the child to sense wetness. However, by making absorbent articles so absorbent, it is difficult for the wearer to realize that an insult of the article has occurred.

To this end, some prior articles intended for personal wear during toilet training include means for alerting a child to urination without leaving a substantial amount of wetness against the skin. One example of training pants intended to provide a sensory indication of urination includes an element that changes size after urination (e.g., expanding upon wetting). However, such elements are typically surrounded by highly absorbent structures (sometimes referred to as absorbent cores) which compete for and may draw urine away from the element, thereby prolonging or otherwise inhibiting the expansion thereof and diminishing its potential training effectiveness. Also, superabsorbent material (SAM) which is used to make the highly absorbent structures of such articles expands upon absorbing urine. Such expansion may mask or otherwise cushion the feeling of the expanded sensory element, thus making it difficult for the wearer to sense the intended signal. Additionally, the expanding element can expand not only in the direction toward the crotch of the wearer, but also can expand in the plane of the article. This expansion in the plane of the absorbent article can result in increased pressure on the saturated absorbent core, making the absorption and retention of the absorbed liquid more difficult. Thus, the absorbing capacity of the absorbent core can be diminished, which may result in unwanted wetness remaining on the skin of the wearer and/or liquid leaking out of the absorbent article.

Consequently, while there has been progress in the design of personal absorbent articles capable of alerting a wearer to a release of liquid body exudates, there continues to be a need for improvements in such articles.

### SUMMARY OF THE INVENTION

Objects and advantages of the invention will be set forth in part in the following description, or may be obvious from the description, or may be learned through practice of the invention.

In general, the present disclosure is directed toward absorbent articles configured to alert a wearer of a liquid insult. The absorbent article generally includes a liquid-permeable layer, a liquid-impermeable layer, an absorbent core, and a compressed wetness indicator. The compressed wetness indicator is positioned between the liquid-permeable layer and the liquid-impermeable layer. The compressed wetness indicator is generally constructed from a compression molded web and is configured to expand in the z-direction upon contact with a liquid without substantially expanding in either the x-direction or the y-direction. In one particular embodiment, the compression molded web can include a physiological cooling agent.

In another embodiment, the present invention is directed to a method of alerting a wearer of an absorbent article that an insult of the absorbent article has occurred. The absorbent article is placed in contact with the wearer such that upon wetting the compressed wetness indicator in the absorbent article with a bodily fluid, the compressed wetness indicator expands 1-dimensionally in a direction toward the wearer, wherein the compressed wetness indicator expands according to an expansion ratio of greater than about 2:1.1.

Other features and aspects of the present invention are discussed in greater detail below.

### BRIEF DESCRIPTION OF THE DRAWINGS

A full and enabling disclosure of the present invention, including the best mode thereof to one skilled in the art, is set forth more particularly in the remainder of the specification, which includes reference to the accompanying figures, in which:

FIGS. 1A, 1C, and 1E shown exemplary wetness indicators in its compressed states;

FIGS. 1B, 1D, and 1F respectfully show the exemplary wetness indicators of FIGS. 1A, 1C, and 1E in their expanded states;

FIGS. 2A and 2B show an exemplary absorbent article including a wetness indicator in both its compressed and expanded states, respectfully;

FIG. 3 shows the construction of an exemplary diaper including a wetness indicator according to one embodiment of the present invention;

FIG. 4 shows an exemplary training pant including a wetness indicator according to one embodiment of the present invention;

FIG. 5 shows an exemplary sanitary napkin for feminine care including a wetness indicator according to one embodiment of the present invention; and

FIG. 6 is a chart plotting the expansion of an exemplary compressed wetness indicator in the z-direction as a function of the amount of water it contacts.

Repeat use of reference characters in the present specification and drawings is intended to represent the same or analogous features or elements of the present invention.

### DETAILED DESCRIPTION

Reference now will be made to the embodiments of the invention, one or more examples of which are set forth below. Each example is provided by way of an explanation of the invention, not as a limitation of the invention. In fact, it will be apparent to those skilled in the art that various modifications and variations can be made in the invention without departing from the scope or spirit of the invention. For instance, features illustrated or described as one embodiment can be used on another embodiment to yield still a further embodiment. Thus, it is intended that the present invention cover such modifications and variations as come within the scope of the appended claims and their equivalents. It is to be understood by one of ordinary skill in the art that the present discussion is a description of exemplary embodiments only, and is not intended as limiting the broader aspects of the present invention, which broader aspects are embodied exemplary constructions.

In general, the present disclosure is directed to providing a wetness indicator within absorbent articles. The wetness indicator of the present invention does not substantially alter or interfere with the absorbent capabilities of the absorbent article by pressing against the absorbent core in the x- and y-directions. Thus, the wetness indicator can be included within conventional absorbent articles without significantly sacrificing the absorbency characteristics of the article.

The wetness indicator of the present invention is configured to expand toward the skin of the wearer (i.e., in the z-direction of the absorbent article perpendicular to the plane of the absorbent article) upon contact with a liquid. However, the expansion of the wetness indicator is substantially limited to the z-direction. That is, the wetness indicator does not substantially expand in any direction parallel with the plane of the article (i.e., the x- and y-directions). As such, the wetness indicator does not significantly interfere with the absorbent capabilities of the absorbent article.

### I. Wetness Indicator

According to the present invention, the wetness indicator is constructed from a highly compressed web material. After compression-molding of the web material, a compressed wetness indicator is formed that is configured to expand only in the direction of the compression forces (i.e., only in the z-direction) upon wetting. Thus, the direction of expansion upon contact with a liquid can be predisposed, allowing the direction of expansion of the compressed wetness indicator to be predetermined when included within an absorbent article.

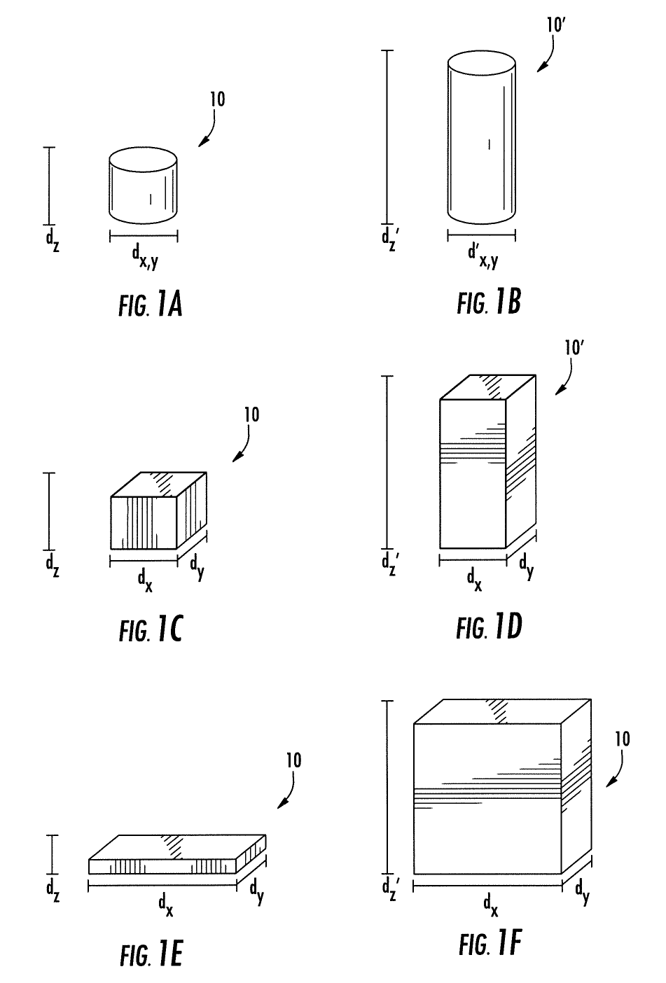

Referring to FIG. 1A, an exemplary compressed wetness indicator 10 is shown in its dry, compressed state. The compressed wetness indicator 10 has a compressed height d_{z} in its z-direction while still in its dry state. Upon contact with a liquid, the compressed wetness indicator 10 expands to be an expanded wetness indicator 10′ having an expanded height d_{z}′ (as shown in FIG. 1B). The degree of expansion in the z-direction can be predetermined by the type of material included within the compressed wetness indicator 10 and the force asserted in forming the compressed wetness indicator 10.

The expansion of the compressed wetness indicator 10 is substantially 1-dimensional. Upon contact with a liquid expansion of the compressed wetness indicator 10 occurs in the z-direction, without substantially increasing the size of the compressed wetness indicator 10 in either the x-direction or y-direction. For example, referring to FIGS. 1A and 1B, the compressed wetness indicator 10 is shown having a cylindrical shape, such that its size in the x- and y-directions are substantially equal (i.e., the diameter of the cylindrical wetness indicator 10). The diameter d_{x,y} of the compressed wetness indicator 10 remains substantially unchanged after contact with a liquid causing expansion in the z-direction. Thus, the diameter d_{x,y}′ of the expanded wetness indicator 10′ shown in FIG. 1B is nearly identical to the diameter d_{x,y} of the compressed wetness indicator 10 shown in FIG. 1A (e.g., d_{x,y}′≦1.1 times d_{x,y}).

The expansion of the compressed wetness indicator can be stated as an “expansion ratio” comparing of the degree of expansion in the z-direction compared to the degree of expansion in both the x- and y-directions (i.e., d_{z}′ divided by d_{z} compared to d_{x,y}′ divided by d_{x,y}). In particular embodiments, the compressed wetness indicator can expand more than about 2:1.1 in the z-direction compared to the x- and y-directions, such as greater than 3:1.1, and from about 5:1.1 to about 10:1.1. For example, the expansion ration can be greater than about 2:1.05, such as greater than about 3:1.05, such as from about 5:1.05 to about 10:1.05.

For example, the compressed wetness indicator 10 suitably expands to at least about 2 times its original height d_{z} in the z-direction when dry (i.e., expands 200%), and more suitably it expands to at least about 3 times the original height d_{z} when dry (i.e., expands 300%). For example, in some embodiments, the expanded wetness indicator 10′ can have a thickness or height d_{z}′ that is from about 5 times to about 10 times its original height d_{z} (i.e., expands from about 500% to about 1000%).

In one particular embodiment, the diameter d_{x,y}′ of the expanded wetness indicator 10′ can be less than about 110% of the diameter d_{x,y} of the compressed wetness indicator 10 in a dry state (i.e., less than about 1.1 times the original diameter d_{x,y}), such as from 100% (i.e., unchanged in diameter upon contact with a liquid in the x- and y-directions) to about 107% (i.e., about 1.07 times the original diameter d_{x,y}). For instance, the diameter d_{x,y}′ of the expanded wetness indicator 10′ can be from about 100.5% to about 105% of the diameter d_{x,y} of the compressed wetness indicator 10 in a dry state.

Of course, the compressed wetness indicator 10 can be molded into any other shape, including but not limited to cuboids, cubes, cones, etc. No matter the particular shape of the compressed wetness indicator 10, the dimensions in the x- and y-directions do not substantially increase upon contact with a liquid.

For example, referring to FIG. 1C, a cube-shaped compressed wetness indicator 10 is shown having substantially equal dimensions in the z-direction (d_{z}), the x-direction (dₓ), and the y-direction (d_{y}). Upon contact with a liquid, the cube-shaped compressed wetness indicator 10 expands one-dimensionally in the z-direction, as shown in FIG. 1D. In another embodiment, the compressed wetness indicator 10 can be a rectangular box, as shown in FIG. 1E. In the shown embodiment, the rectangular box-shaped compressed wetness indicator 10 has a height in the z-direction (d_{z}), a length in the x-direction (dₓ), and a width in the y-direction (d_{y}). Upon contact with a liquid, the rectangular box-shaped compressed wetness indicator 10 expands one-dimensionally in the z-direction, as shown in FIG. 1F. The expansion of these embodiments is substantially similar to that described above with reference to the cylindrically-shaped compressed wetness indicator 10.

The compressed wetness indicator 10 is configured to expand to the expanded wetness indicator 10′ nearly immediately upon contact with a small amount of a liquid. For example, the 1-dimensional expansion can occur within about 10 seconds of the compressed wetness indicator 10 contacting a liquid, such expanding in less than about 5 seconds. In some embodiments, the 1-dimensional expansion of the compressed wetness indicator 10 can occur from about 1 second to about 5 seconds, such as from about 1 second to about 3 seconds. Thus, the wearer of the absorbent article can be immediately alerted upon the first insult of the absorbent article.

In order to initiate the expansion of the compressed wetness indicator 10, the compressed wetness indicator 10 is configured to expand upon contact with a small amount of liquid. This amount of liquid need not completely saturate the compressed wetness indicator 10. Of course, the amount of liquid necessary to cause complete expansion of the compressed wetness indicator 10 to the expanded wetness indicator 10′ can vary with the size of the compressed wetness indicator 10. However, when used in an absorbent article, the compressed wetness indicator 10 is configured, in most embodiments, to expand upon contact with greater than about 0.1 milliliters (mL), such as from about 0.5 mL to about 7 mL, and from about 1 mL to about 5 mL. At these liquid levels, the compressed wetness indicator 10 can at least double in height in the z-direction with an expansion ratio of at least 2:1.1, as stated above.

The wetness indicator offer the moisture triggered z-directional expansion with a significant amount of energy. Specifically, the wetness indicator can expand in the z-direction with an exerted force up to about 16 pounds per square inch (psi), such from about 10 psi to about 15 psi. Thus, the wetness indicator can press against the skin of the wearer with sufficient force to alert the wearer that an insult has occurred.

The web material that is compressed to form the wetness indicator can be a nonwoven web of fibers. Although the particular type of fiber is not a limitation of the invention, some fibers are particularly suitable for forming the compressed wetness indicator 10 to be included within an absorbent article. The fibers may be, for example, any combination of synthetic or pulp fibers. The selected average fiber length and denier will generally depend on a variety of factors and desired processing steps.

In one embodiment, a substantial portion of the fibers may be cellulosic pulp staple fibers. Pulp fibers may be utilized to reduce costs, as well as impart other benefits to the compressed wetness indicator 10, such as improved absorbency. Some examples of suitable cellulosic fiber sources include virgin wood fibers, such as thermomechanical, bleached and unbleached pulp fibers. Pulp fibers may have a high-average fiber length, a low-average fiber length, or mixtures of the same. Some examples of suitable high-average length pulp fibers include northern softwood, southern softwood, redwood, red cedar, hemlock, pine (e.g., southern pines), spruce (e.g., black spruce), combinations thereof, and so forth. Some examples of suitable low-average fiber length pulp fibers may include certain virgin hardwood pulps and secondary (i.e. recycled) fiber pulp from sources such as, for example, newsprint, reclaimed paperboard, and office waste. Hardwood fibers, such as eucalyptus, maple, birch, aspen, and so forth, may also be used as low-average length pulp fibers. These pulp fibers can be formed into a nonwoven web (e.g., a tissue web) according to any process (e.g., wetlaid, airlaid, bonded carded process, etc.).

In one particular embodiment, the web is a non-woven web of rayon material. In particular, the rayon material can be manufactured by a spun lace method in which a web is formed out of viscose rayon and fibers are coupled using a high-pressure water stream.

Alternatively, a majority of the fibers of the nonwoven web may be formed from synthetic polymers. Synthetic fibers can be formed into nonwoven fabrics or webs from many processes such as for example, meltblowing processes, spunbonding processes, bonded carded web processes, etc.

“Meltblown fibers” refers to fibers formed by extruding a molten thermoplastic material through a plurality of fine, usually circular, die capillaries as molten fibers into converging high velocity gas (e.g. air) streams that attenuate the fibers of molten thermoplastic material to reduce their diameter, which may be to microfiber diameter. Thereafter, the meltblown fibers are carried by the high velocity gas stream and are deposited on a collecting surface to form a web of randomly disbursed meltblown fibers. Such a process is disclosed, for example, in U.S. Pat. No. 3,849,241 to Butin, et al., which is incorporated herein in its entirety by reference thereto for all purposes. Generally speaking, meltblown fibers may be microfibers that may be continuous or discontinuous, are generally smaller than 10 microns in diameter, and are generally tacky when deposited onto a collecting surface.

“Spunbonded fibers” refers to small diameter substantially continuous fibers that are formed by extruding a molten thermoplastic material from a plurality of fine, usually circular, capillaries of a spinnerette with the diameter of the extruded fibers then being rapidly reduced as by, for example, eductive drawing and/or other well-known spunbonding mechanisms. The production of spun-bonded nonwoven webs is described and illustrated, for example, in U.S. Pat. Nos. 4,340,563 to Appel, et al., 3,692,618 to Dorschner, et al., 3,802,817 to Matsuki, et al., 3,338,992 to Kinney, 3,341,394 to Kinney, 3,502,763 to Hartman, 3,502,538 to Petersen, 3,542,615 to Dobo, et al., and 5,382,400 to Pike, et al., which are incorporated herein in their entirety by reference thereto for all purposes. Spunbond fibers are generally not tacky when they are deposited onto a collecting surface. Spunbond fibers can sometimes have diameters less than about 40 microns, and are often between about 5 to about 20 microns.

Exemplary synthetic polymers for use in forming nonwoven web may include, for instance, polyolefins, e.g., polyethylene, polypropylene, polybutylene, etc.; polytetrafluoroethylene; polyesters, e.g., polyethylene terephthalate and so forth; polyvinyl acetate; polyvinyl chloride acetate; polyvinyl butyral; acrylic resins, e.g., polyacrylate, polymethylacrylate, polymethylmethacrylate, and so forth; polyamides, e.g., nylon; polyvinyl chloride; polyvinylidene chloride; polystyrene; polyvinyl alcohol; polyurethanes; polylactic acid; copolymers thereof; and so forth. If desired such as those described above, may also be employed. It should be noted that the polymer(s) may also contain other additives, such as processing aids or treatment compositions to impart desired properties to the fibers, residual amounts of solvents, pigments or colorants, and so forth.

Monocomponent and/or multicomponent fibers may be used to form the nonwoven web. Monocomponent fibers are generally formed from a polymer or blend of polymers extruded from a single extruder. Multicomponent fibers are generally formed from two or more polymers (e.g., bicomponent fibers) extruded from separate extruders. The polymers may be arranged in substantially constantly positioned distinct zones across the cross-section of the fibers. The components may be arranged in any desired configuration, such as sheath-core, side-by-side, pie, island-in-the-sea, three island, bull's eye, or various other arrangements known in the art. Various methods for forming multicomponent fibers are described in U.S. Pat. Nos. 4,789,592 to Taniguchi et al. and U.S. Pat. No. 5,336,552 to Strack, et al., 5,108,820 to Kaneko, et al., 4,795,668 to Kruege, et al., 5,382,400 to Pike, et al., 5,336,552 to Strack. et al., and 6,200,669 to Marmon. et al., which are incorporated herein in their entirety by reference thereto for all purposes. Multicomponent fibers having various irregular shapes may also be formed, such as described in U.S. Pat. Nos. 5,277,976 to Hogle, et al., 5,162,074 to Hills, 5,466,410 to Hills, 5,069,970 to Largman, et al., and 5,057,368 to Largman, et al., which are incorporated herein in their entirety by reference thereto for all purposes.

Although any combination of polymers may be used, the polymers of the multicomponent fibers are typically made from thermoplastic materials with different glass transition or melting temperatures where a first component (e.g., sheath) melts at a temperature lower than a second component (e.g., core). Softening or melting of the first polymer component of the multicomponent fiber allows the multicomponent fibers to form a tacky skeletal structure, which upon cooling, stabilizes the fibrous structure. For example, the multicomponent fibers may have from about 5% to about 80%, and in some embodiments, from about 10% to about 60% by weight of the low melting polymer. Further, the multicomponent fibers may have from about 95% to about 20%, and in some embodiments, from about 90% to about 40%, by weight of the high melting polymer. Some examples of known sheath-core bicomponent fibers available from KoSa Inc. of Charlotte, N.C. under the designations T-255 and T-256, both of which use a polyolefin sheath, or T-254, which has a low melt co-polyester sheath. Still other known bicomponent fibers that may be used include those available from the Chisso Corporation of Moriyama, Japan or Fibervisions LLC of Wilmington, Del.

Fibers of any desired length may be employed, such as staple fibers, continuous fibers, etc. In one particular embodiment, for example, staple fibers may be used that have a fiber length in the range of from about 1 to about 150 millimeters, in some embodiments from about 5 to about 50 millimeters, in some embodiments from about 10 to about 40 millimeters, and in some embodiments, from about 10 to about 25 millimeters. Although not required, carding techniques may be employed to form fibrous layers with staple fibers as is well known in the art. For example, fibers may be formed into a carded web by placing bales of the fibers into a picker that separates the fibers. Next, the fibers are sent through a combing or carding unit that further breaks apart and aligns the fibers in the machine direction so as to form a machine direction-oriented fibrous nonwoven web. The carded web may then be bonded using known techniques to form a bonded carded nonwoven web.

If desired, the nonwoven web may have a multi-layer structure. The other layers can be other nonwoven webs, films, and the like. For example, in one embodiment, at least two nonwoven webs can be combined to form a nonwoven laminate. Suitable multi-layered materials may include, for instance, spunbond/meltblown/spunbond (SMS) laminates and spunbond/meltblown (SM) laminates. Various examples of suitable SMS laminates are described in U.S. Pat. Nos. 4,041,203 to Brock et al.; 5,213,881 to Timmons, et al.; 5,464,688 to Timmons, et al.; 4,374,888 to Bornslaeger; 5,169,706 to Collier, et al.; and 4,766,029 to Brock et al., which are incorporated herein in their entirety by reference thereto for all purposes. In addition, commercially available SMS laminates may be obtained from Kimberly-Clark Corporation under the designations Spunguard® and Evolution®).

Another example of a multi-layered structure is a spunbond web produced on a multiple spin bank machine in which a spin bank deposits fibers over a layer of fibers deposited from a previous spin bank. Such an individual spunbond nonwoven web may also be thought of as a multi-layered structure. In this situation, the various layers of deposited fibers in the nonwoven web may be the same, or they may be different in basis weight and/or in terms of the composition, type, size, level of crimp, and/or shape of the fibers produced. As another example, a single nonwoven web may be provided as two or more individually produced layers of a spunbond web, a carded web, etc., which have been bonded together to form the nonwoven web. These individually produced layers may differ in terms of production method, basis weight, composition, and fibers as discussed above.

A nonwoven web constructed from synthetic fibers may also contain an additional fibrous component such that it is considered a composite. For example, a nonwoven web may be entangled with another fibrous component using any of a variety of entanglement techniques known in the art (e.g., hydraulic, air, mechanical, etc.). In one embodiment, the nonwoven web is integrally entangled with cellulosic fibers using hydraulic entanglement. Hydraulically entangled nonwoven webs of staple length and continuous fibers are disclosed, for example, in U.S. Pat. Nos. 3,494,821 to Evans and 4,144,370 to Boulton, which are incorporated herein in their entirety by reference thereto for all purposes. Hydraulically entangled composite nonwoven webs of a continuous fiber nonwoven web and a pulp layer are disclosed, for example, in U.S. Pat. Nos. 5,284,703 to Everhart. et al. and 6,315,864 to Anderson, et al., which are incorporated herein in their entirety by reference thereto for all purposes.

No matter the particular construction of the nonwoven web, the web is compression molded into a compressed wetness indicator 10 configured to expand 1-dimensionally. The 1-dimensional expansion generally occurs in the direction of the compression forces exerted during the formation of the compressed wetness indicator 10. Thus, one of ordinary skill in the art would be able to form a compressed wetness indicator 10 having any desired shape and any desired expansion parameters.

In one embodiment, the compressed web materials can be formed by first folding or rolling the web material into a tube-like shape, such that the web material is generally longer in the z-direction than in the x- and y-directions. This folded or rolled web material is then placed into an elongated barrel such that the longer z-direction of the folded or rolled web is parallel with the length of the barrel. The shape of the barrel in the x- and y-directions corresponds to the shape of the resulting compressed wetness indicator 10. For example, to make the compressed wetness indicator 10 shown in FIG. 1A, the barrel shape is cyclical such that the x- and y- directions of the barrel define a circle (or oval). Alternatively, the barrel shape can define any desired shape in the x- and y-directions to produce the compressed wetness indicator 10 in the desired shape.

After placement in the barrel, the folded or rolled web is subjected to a compression force in a direction of the elongation of the barrel (i.e., the z-direction). This compression force is sufficient to compress the folded or rolled web into a compressed wetness indicator 10 that will not retain its initial shape until after exposure to a liquid. That is, the disposable tissue 1 of the present invention should be subjected to compression molding under a pressure within a predetermined pressure range that varies according to the shape, configuration, and chemical construction of the web as described above. However, if the web is pressed under a pressure within the predetermined pressure range, it is compressed at a compressibility (ΔV/V) in a range of 0.4 to 0.6. Here, the compressibility (ΔV/V) represents a ratio of the amount of volume change (ΔV) in the compressed wetness indicator 10 to the volume (V) of the uncompressed web. The amount of volume change means the difference between the volume (V) of the uncompressed web and the volume of the compressed wetness indicator 10.

For example, when making a compressed wetness indicator 10 shaped as in FIG. 1A with a diameter d_{x,y} of about 2 cm and a height d_{z} of about 1 cm from a web. The web can have any initial size, such as less than about 20 cm×20 cm, such as from about 5 cm×5 cm to about 15 cm×15 cm. In one particular embodiment, the web can have an initial size of about 10×10 cm. The compression force can be apply a pressure to the folded or rolled tissue web of about 95 kiloNewton (kN) to about 300 kN, such as from about 145 kN to about 250 kN. In one particular embodiment the compression force can be from about 190 kN to about 200 kN in the z-direction.

Although the apparatus for forming the compressed wetness indicator 10 can vary, a particularly suitable apparatus can include a cylindrical molding barrel having a longitudinal, through passage. The molding barrel can be supported on a table such that both end portions of the through passage of the molding barrel are exposed to the outside. An upper press can be installed vertically movably above the table and having a pressing rod to be inserted into the through passage of the molding barrel when the upper press moves downwardly. A lower press can also be installed vertically movably below the table and having a supporting rod to be inserted into the through passage of the molding barrel when the lower press moves upwardly.

In this set up, the upper press can include a power source for pressing the folded or rolled web received in the through passage. The supporting rod of the lower press closes an entrance of the through passage of the molding barrel to compression-mold the folded or rolled web and opens the entrance of the through passage to discharge the compressed wetness indicator 10 from the through passage. The compressed wetness indicator 10 is molded to have a shape that is the same as a space defined by the through passage of the molding barrel, the supporting rod of the lower press, and the pressing rod of the upper press. In a state where the entrance of the through passage of the molding barrel is opened, the compressed wetness indicator 10 is discharged from the through passage by the upper press moving downwardly.

In one particular embodiment, the compressed web materials can be made with the compression molding apparatus and methods described in International Publication No. WO 200/082448 A1 of Lee, et al., the disclosure of which is incorporated herein by reference.

In one particular embodiment, a physiological cooling agent can be included on or in the compressed wetness indicator 10 to provide an additional tactile cue in the form of a cooling sensation to the skin of the wearer upon contact. Thus, when the compressed wetness indicator 10 expands upon contact with a liquid, the expanded wetness indicator 10′ pressed to the skin of the wearer can create a cooling sensation, further alerting the wearer that an insult of the absorbent article has occurred. For example, the cooling sensation chemical can be added to the side of the wetness indicator that is configured to press against the skin of the wearer upon wetting.

The physiological cooling agent can be, in one embodiment, a polyol. Many polyols are known to provide a cooling sensation upon contact with skin due to their endothermic (heat-absorbing) reaction when dissolving in moisture (e.g., the liquid insulting the absorbent article, the moisture located on the skin, etc.). Suitable polyols can include those of a hydrogenated form of carbohydrate, whose carbonyl group (aldehyde or ketone, reducing sugar) has been reduced to a primary or secondary hydroxyl group. These polyols can have a general formula H(HCOH)ₙ₊₁H, whereas sugar's is H(HCOH)ₙHCO, where n is an integer from 0 to 10. Exemplary polyols can include, but are not limited to, glycol, glycerol, erythritol, arabitol, xylitol, zylitol, mannitol, sorbitol, and the like. The use of such a physiological cooling agent can provide a wetness sensation on the skin of the wearer without actual moisture remaining on the skin.

In yet another tactile cue, a tingling sensation can be created on the skin of the wearer upon contact with a liquid. The tingling sensation is generated from gas formation, such as disclosed in U.S. Pat. No. 6,929,819 of Underhill, et al., which is incorporated herein. Generally, the wetness indicator can include an effervescent agent or combination of agents that alerts the wearer that urination has occurred by releasing gas and causing a mild concussive (i.e., “popping,” “crackling,” “bubbling” or “fizzing”) sensation on or next to the wearer's skin upon urination. This may be accomplished without trapping moisture against the skin of the wearer. One example of a suitable acid/base combination is shown in equation (1)

NaHCO₃+KHC₄H₄O₆→KNaC₄H₄O₆+H₂O+CO₂ (1)

In equation (1), sodium bicarbonate and potassium bitartrate react in the presence of a liquid (urine) to form carbon dioxide gas and by-products. The production of the carbon dioxide gas alerts the wearer of the pad containing the acid and base that urination has occurred.

Another example of a suitable acid/base combination is shown in equation (2):

NaAl(SO₄)₂+3NaHCO₃→Al(OH)₃+2Na₂SO₄+3CO₂ (2)

In equation (2), sodium aluminum sulfate and sodium bicarbonate react in the presence of liquid (urine) to form carbon dioxide gas and by-products. Other acids that can be used in combination with sodium bicarbonate to produce an effervescent agent in accordance with the present invention include ascorbic, lactic, glycolic, malic, tartaric, and fumaric. When mixed with sodium bicarbonate and contacted with urine, these acids produce carbon dioxide gas. This gas production can alert the wearer that urination has occurred.

### II. Absorbent Articles

An “absorbent article” generally refers to any article capable of absorbing water or other fluids. Examples of some absorbent articles include, but are not limited to, personal care absorbent articles, such as diapers, training pants, absorbent underpants, incontinence articles, feminine hygiene products (e.g., sanitary napkins), swim wear, baby wipes, and so forth; medical absorbent articles, such as garments, fenestration materials, underpads, bedpads, bandages, absorbent drapes, and medical wipes; food service wipers; clothing articles; and so forth. Materials and processes suitable for forming such absorbent articles are well known to those skilled in the art. Typically, absorbent articles include a substantially liquid-impermeable layer (e.g., outer cover), a liquid-permeable layer (e.g., bodyside liner, surge layer, etc.), and an absorbent core.

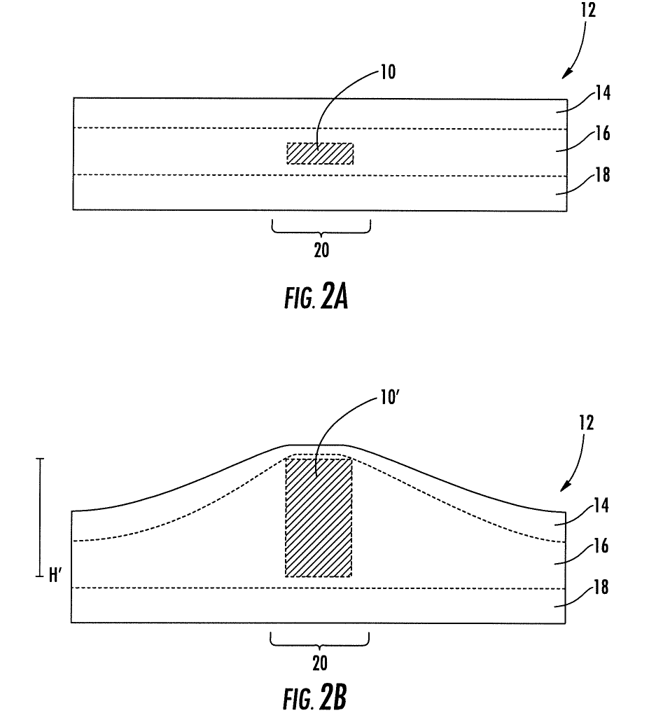

With particular reference now to FIG. 2A, a compressed wetness indicator is suitably disposed between the liquid-permeable layer 14 and the liquid-impermeable layer 18 so that the compressed wetness indicator 10 is substantially imperceptible to the wearer prior to the first insult of the absorbent article 12 by liquid body exudates (e.g., urine, menses, feces). As shown, the compressed wetness indicator 10 is positioned in the middle crotch region 20 of the absorbent article 12 (e.g., within the middle third of the absorbent article in both the longitudinal and lateral directions). However, it is contemplated that the longitudinal position of the compressed wetness indicator 10 within the crotch region 20 (e.g., the middle third of the length of the absorbent article 12) may be dependant on the type of absorbent article and/or the gender of the intended wearer.

The compressed wetness indicator 10 may be disposed in the construction of the absorbent core 16 other than between the liquid-permeable layer 14 and the liquid-impermeable layer 18. For example, the compressed wetness indicator 10 may be disposed on the liquid-permeable layer 14 for direct contact with the wearer's skin and remain within the scope of this invention. In such an embodiment, the compressed wetness indicator 10 may be attached to the liquid-permeable layer 14, such as by being adhesively attached or bonded thereto, or it may be releasably secured to the liquid-permeable layer 14 to permit the caregiver to position the compressed wetness indicator 10 on the liquid-permeable layer 14 in a desired longitudinal position depending on the type of absorbent article and/or the gender of the wearer.

While a single compressed wetness indicator 10 is shown in the illustrated embodiment of FIG. 2A, it is contemplated that additional compressed wetness indicators 10 may be used to further enhance the signal to the wearer. For example, additional compressed wetness indicator 10 may be necessary for larger absorbent articles for whom the resistive force provided by a single compressed wetness indicator 10 may be insufficient to alert the wearer to insult of the absorbent article 12.

The thickness, or height H, of the compressed wetness indicator 10 when dry is suitably in the range of about 2 mm to about 20 mm, and more suitably in the range of about 5 mm to about 15 mm, such as about 10 mm. Upon absorption of a liquid, the thickness, or height H′, of the expanded wetness indicator 10′ suitably expands to at least about 2 times its original height H when dry, and more suitably it expands to at least about 3 times the height H when dry. For example, in some embodiments, the expanded wetness indicator 10′ can have a thickness or height H′ that is from about 5 times to about 10 times its original height H. This 1-dimensional expansion is generally achieved according to the expansion ratio described above, with contact of greater than 0.1 mL of a liquid.

At the relatively small initial height H, the compressed wetness indicator 10 does not substantially interfere with the flexibility of the absorbent article, nor does the compressed wetness indicator 10 substantially interfere with the absorbent capacity of the absorbent core 16. For example, the compressed wetness indicator 10 can have a width of less than about 33% of the width of the absorbent core, such as less than about 25%. In most embodiments, the compressed wetness indicator 10 has a width and length in the x- and y- directions of less than about 5 centimeters (cm), such as from about 1 cm to about 4 cm, and from about 2 cm to about 3 cm.

Various embodiments of an absorbent article that may be formed according to the present invention will now be described in more detail. However, as noted above, the invention may be embodied in any type of absorbent articles, such as diapers, incontinence articles, sanitary napkins, diaper pants, feminine napkins, children's training pants, and so forth.

A. Diapers, Training Pants, and Incontinent Articles

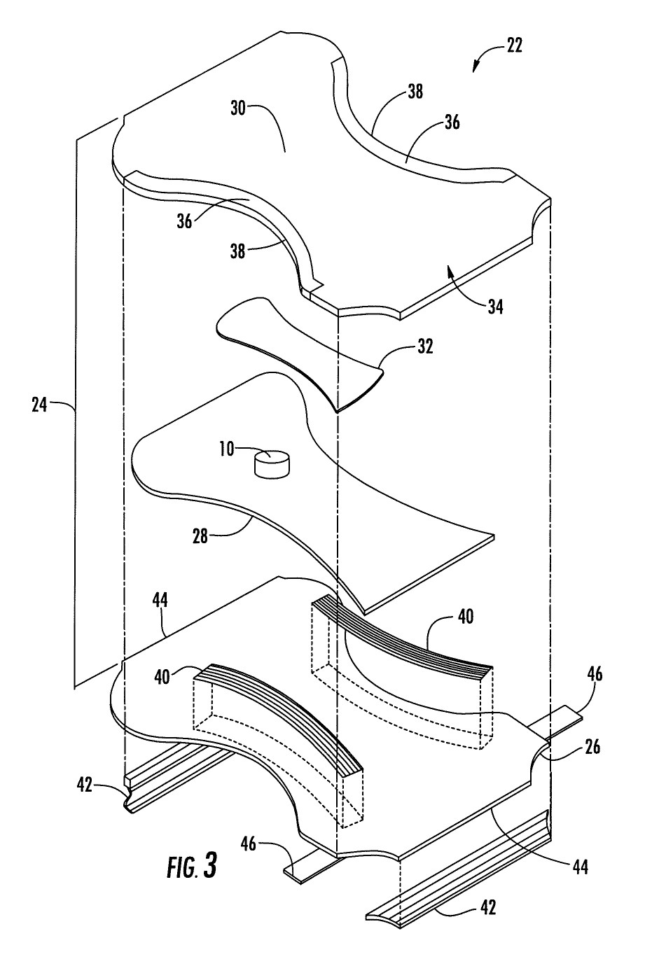

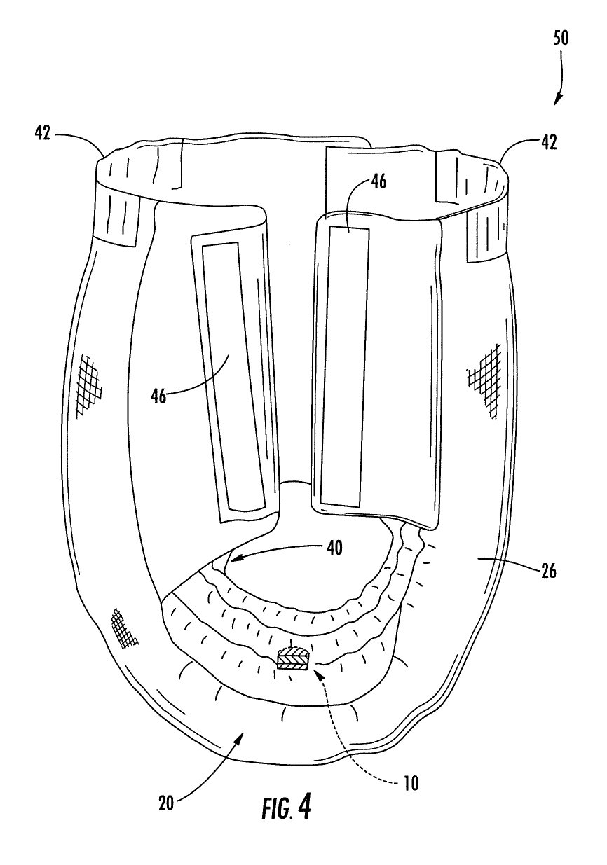

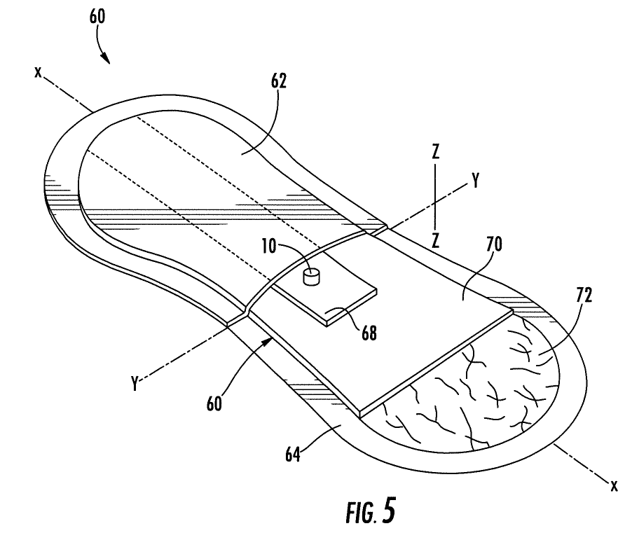

For purposes of illustration only, an absorbent article is shown in FIG. 3 as a diaper 22. In the illustrated embodiment, the diaper 22 is shown as having an hourglass shape in an unfastened configuration. However, other shapes may of course be utilized, such as a generally rectangular shape, T-shape, or I-shape. As shown, the diaper 22 includes a chassis 24 formed by various components, including an outer cover 26, bodyside liner 30, absorbent core 28, and surge layer 32. It should be understood, however, that other layers may also be used in the present invention. Likewise, one or more of the layers referred to in FIG. 3 may also be eliminated in certain embodiments of the present invention.

The outer cover 26 is typically formed from a material that is substantially impermeable to liquids. For example, the outer cover 26 may be formed from a thin plastic film or other flexible liquid-impermeable material. In one embodiment, the outer cover 26 is formed from a polyethylene film having a thickness of from about 0.01 millimeter to about 0.05 millimeter. If a more cloth-like feeling is desired, the outer cover 26 may be formed from a polyolefin film laminated to a nonwoven web. For example, a stretch-thinned polypropylene film having a thickness of about 0.015 millimeter may be thermally laminated to a spunbond web of polypropylene fibers. The polypropylene fibers may have a denier per filament of about 1.5 to 2.5, and the nonwoven web may have a basis weight of about 17 grams per square meter. The outer cover 26 may also include bicomponent fibers, such as polyethylene/polypropylene bicomponent fibers. In addition, the outer cover 26 may also contain a material that is impermeable to liquids, but permeable to gases and water vapor (i.e., “breathable”). This permits vapors to escape from the absorbent core 28, but still prevents liquid exudates from passing through the outer cover 26.

The diaper 22 also includes a bodyside liner 30. The bodyside liner 30 is generally employed to help isolate the wearer's skin from liquids held in the absorbent core 28. For example, the liner 30 presents a bodyfacing surface that is typically compliant, soft feeling, and non-irritating to the wearer's skin. Typically, the liner 30 is also less hydrophilic than the absorbent core 28 so that its surface remains relatively dry to the wearer. The liner 30 may be liquid-permeable to permit liquid to readily penetrate through its thickness. The bodyside liner 30 may be formed from a wide variety of materials, such as porous foams, reticulated foams, apertured plastic films, natural fibers (e.g., wood or cotton fibers), synthetic fibers (e.g., polyester or polypropylene fibers), or a combination thereof. In some embodiments, woven and/or nonwoven fabrics are used for the liner 30. For example, the bodyside liner 30 may be formed from a meltblown or spunbonded web of polyolefin fibers. The liner 30 may also be a bonded-carded web of natural and/or synthetic fibers. The liner 30 may further be composed of a substantially hydrophobic material that is optionally treated with a surfactant or otherwise processed to impart a desired level of wettability and hydrophilicity. The surfactant may be applied by any conventional method, such as spraying, printing, brush coating, foaming, and so forth. When utilized, the surfactant may be applied to the entire liner 30 or may be selectively applied to particular sections of the liner 30, such as to the medial section along the longitudinal centerline of the diaper. The liner 30 may further include a composition that is configured to transfer to the wearer's skin for improving skin health. Suitable compositions for use on the liner 30 are described in U.S. Pat. No. 6,149,934 to Krzysik et al., which is incorporated herein in its entirety by reference thereto for all purposes.

As illustrated in FIG. 3, the diaper 22 may also include a surge layer 32 that helps to decelerate and diffuse surges or gushes of liquid that may be rapidly introduced into the absorbent core 28. Desirably, the surge layer 32 rapidly accepts and temporarily holds the liquid prior to releasing it into the storage or retention portions of the absorbent core 28. In the illustrated embodiment, for example, the surge layer 32 is interposed between an inwardly facing surface of the bodyside liner 30 and the absorbent core 28. Alternatively, the surge layer 32 may be located on an outwardly facing surface 34 of the bodyside liner 30. The surge layer 32 is typically constructed from highly liquid-permeable materials. Suitable materials may include porous woven materials, porous nonwoven materials, and apertured films. Some examples include, without limitation, flexible porous sheets of polyolefin fibers, such as polypropylene, polyethylene or polyester fibers; webs of spunbonded polypropylene, polyethylene or polyester fibers; webs of rayon fibers; bonded carded webs of synthetic or natural fibers or combinations thereof. Other examples of suitable surge layers 32 are described in U.S. Pat. Nos. 5,486,166 to Ellis, et al. and 5,490,846 to Ellis, et al., which are incorporated herein in their entirety by reference thereto for all purposes.

Besides the above-mentioned components, the diaper 22 may also contain various other components as is known in the art. For example, the diaper 22 may also contain a substantially hydrophilic tissue wrapsheet (not illustrated) that helps maintain the integrity of the fibrous structure of the absorbent core 28. The tissue wrapsheet is typically placed about the absorbent core 28 over at least the two major facing surfaces thereof, and composed of an absorbent cellulosic material, such as creped wadding or a high wet-strength tissue. The tissue wrapsheet may be configured to provide a wicking layer that helps to rapidly distribute liquid over the mass of absorbent fibers of the absorbent core 28. The wrapsheet material on one side of the absorbent fibrous mass may be bonded to the wrapsheet located on the opposite side of the fibrous mass to effectively entrap the absorbent core 28.

Furthermore, the diaper 22 may also include a ventilation layer (not shown) that is positioned between the absorbent core 28 and the outer cover 26. When utilized, the ventilation layer may help insulate the outer cover 26 from the absorbent core 28, thereby reducing dampness in the outer cover 26. Examples of such ventilation layers may include breathable laminates (e.g., nonwoven web laminated to a breathable film), such as described in U.S. Pat. No. 6,663,611 to Blaney, et al., which is incorporated herein in its entirety by reference thereto for all purpose.

As representatively illustrated in FIG. 3, the diaper 22 may also include a pair of containment flaps 36 that are configured to provide a barrier and to contain the lateral flow of body exudates. The containment flaps 36 may be located along the laterally opposed side edges 38 of the bodyside liner 30 adjacent the side edges of the absorbent core 28. The containment flaps 36 may extend longitudinally along the entire length of the absorbent core 28, or may only extend partially along the length of the absorbent core 28. When the containment flaps 36 are shorter in length than the absorbent core 28, they may be selectively positioned anywhere along the side edges 38 of diaper 22 in a crotch region 10. In one embodiment, the containment flaps 36 extend along the entire length of the absorbent core 28 to better contain the body exudates. Such containment flaps 36 are generally well known to those skilled in the art. For example, suitable constructions and arrangements for the containment flaps 36 are described in U.S. Pat. No. 4,704,116 to Enloe, which is incorporated herein in its entirety by reference thereto for all purposes.

The diaper 22 may include various elastic or stretchable materials, such as a pair of leg elastic members 40 affixed to the side edges 38 to further prevent leakage of body exudates and to support the absorbent core 28. In addition, a pair of waist elastic members 42 may be affixed to longitudinally opposed waist edges 44 of the diaper 22. The leg elastic members 40 and the waist elastic members 42 are generally adapted to closely fit about the legs and waist of the wearer in use to maintain a positive, contacting relationship with the wearer and to effectively reduce or eliminate the leakage of body exudates from the diaper 22. As used herein, the terms “elastic” and “stretchable” include any material that may be stretched and return to its original shape when relaxed. Suitable polymers for forming such materials include, but are not limited to, block copolymers of polystyrene, polyisoprene and polybutadiene; copolymers of ethylene, natural rubbers and urethanes; etc. Particularly suitable are styrene-butadiene block copolymers sold by Kraton Polymers of Houston, Tex. under the trade name Kraton®). Other suitable polymers include copolymers of ethylene, including without limitation ethylene vinyl acetate, ethylene methyl acrylate, ethylene ethyl acrylate, ethylene acrylic acid, stretchable ethylene-propylene copolymers, and combinations thereof. Also suitable are coextruded composites of the foregoing, and elastomeric staple integrated composites where staple fibers of polypropylene, polyester, cotton and other materials are integrated into an elastomeric meltblown web. Certain elastomeric single-site or metallocene-catalyzed olefin polymers and copolymers are also suitable for the side panels.

The diaper 22 may also include one or more fasteners 46. For example, two flexible fasteners 46 are illustrated in FIG. 3 on opposite side edges of waist regions to create a waist opening and a pair of leg openings about the wearer. The shape of the fasteners 46 may generally vary, but may include, for instance, generally rectangular shapes, square shapes, circular shapes, triangular shapes, oval shapes, linear shapes, and so forth. The fasteners may include, for instance, a hook material. In one particular embodiment, each fastener 46 includes a separate piece of hook material affixed to the inside surface of a flexible backing.

The various regions and/or components of the diaper 22 may be assembled together using any known attachment mechanism, such as adhesive, ultrasonic, thermal bonds, etc. Suitable adhesives may include, for instance, hot melt adhesives, pressure-sensitive adhesives, and so forth. When utilized, the adhesive may be applied as a uniform layer, a patterned layer, a sprayed pattern, or any of separate lines, swirls or dots. In the illustrated embodiment, for example, the outer cover 26 and bodyside liner 30 are assembled to each other and to the absorbent core 28 using an adhesive. Alternatively, the absorbent core 28 may be connected to the outer cover 26 using conventional fasteners, such as buttons, hook and loop type fasteners, adhesive tape fasteners, and so forth. Similarly, other diaper components, such as the leg elastic members 40, waist elastic members 42 and fasteners 46, may also be assembled into the diaper 22 using any attachment mechanism.

Although various configurations of a diaper have been described above, it should be understood that other diaper and absorbent article configurations are also included within the scope of the present invention. For instance, other suitable diaper configurations are described in U.S. Pat. Nos. 4,798,603 to Meyer et al.; 5,176,668 to Bemardin; 5,176,672 to Bruemmer et al.; 5,192,606 to Proxmire et al.; and 5,509,915 to Hanson et al., as well as U.S. Patent Application Pub. No. 2003/120253 to Wentzel, et al., all of which are incorporated herein in their entirety by reference thereto for all purposes.

According to the present invention, the compressed wetness indicator 10 can be positioned between the outer cover 26 and the bodyside liner 30 in the diaper 22. In one particular embodiment, the compressed wetness indicator 10 can be located between the absorbent core 28 and the bodyside liner 30. For example, in the diaper 22 shown in FIG. 3, the compressed wetness indicator 10 is positioned between the absorbent core 28 and the surge layer 32 in the target zone (e.g., the crotch region) of the diaper 22. This particular orientation allows the compressed wetness indicator 10 to contact a sufficient amount of liquid upon insult of the diaper 22, while still dissipating the liquid to the absorbent core 28 for absorption. However, the compressed wetness indicator 10 could be located on either side of the surge layer 32.

Additionally, the lateral placement of the compressed wetness indicator 10 can vary according to the gender of the intended wearer. For example, placement of the compressed wetness indicator 10 in a more forward location within the crotch region 20 may be appropriate for boys, while placement in a more central location within the crotch region 20 may be more appropriate for girls. It is also understood that the compressed wetness indicator 10 may be positioned other than in the crotch region 26 without departing from the scope of the present invention, as long as the wetness indicator is suitably positioned so as to become wet and perceptible by a wearer upon insult of the pants by liquid body exudates.

Alternatively, a pair of compressed wetness indicators 10 may also be used in a configuration wherein one wetness indicator is positioned longitudinally where it is more likely to become wet upon urination by boys and the other wetness indicator is positioned longitudinally where it is more likely to become wet upon urination by girls, thereby accounting for differences between the target wetting areas of boys and girls.

In another embodiment, a training pant 50 can be constructed with a compressed wetness indicator 10 within the crotch region 20. The training pant 50 can have a similar construction than the diaper 22 described above. As stated, the compressed wetness indicator 10 of the illustrated embodiment is small enough to not take up a substantial part of the crotch region.

B. Absorbent Pads

In another embodiment, the compressed wetness indicator 10 can be included within a sanitary napkin 60 for feminine hygiene. However, as discussed above, the compressed wetness indicator 10 may be embodied in other types of feminine hygiene products. Nonetheless, in the illustrated embodiment, the sanitary napkin 60 includes a liner 62, a baffle 64, and an absorbent core 66, between any of which the compressed wetness indicator 10 may be positioned. The absorbent core 66 is positioned inward from the outer periphery of the sanitary napkin 60 and includes a body-facing surface positioned adjacent the liner 62 and a garment-facing surface positioned adjacent the baffle 64.

Not only does the compressed wetness indicator 10 indicate to the wearer that it has been insulted, but also the compressed wetness indicator 10 helps maintain a close, secure fit of the sanitary napkin 60 with the body of the wearer.

The liner 62 is generally designed to contact the body of the user and is liquid-permeable. The liner 62 can surround the absorbent core 66 so that it completely encases the sanitary napkin 60. Alternatively, the liner 62 and the baffle 64 can extend beyond the absorbent core 66 and be peripherally joined together, either entirely or partially, using known techniques. Typically, the liner 62 and the baffle 64 are joined by adhesive bonding, ultrasonic bonding, or any other suitable joining method known in the art.

The liquid-permeable liner 62 is sanitary, clean in appearance, and somewhat opaque to hide bodily discharges collected in and absorbed by the absorbent core 66. The liner 62 further exhibits good strike-through and rewet characteristics permitting bodily discharges to rapidly penetrate through the liner 62 to the absorbent core 66, but not allow the body fluid to flow back through the liner 62 to the skin of the wearer. For example, some suitable materials that can be used for the liner 62 include nonwoven materials, perforated thermoplastic films, or combinations thereof. A nonwoven fabric made from polyester, polyethylene, polypropylene, bicomponent, nylon, rayon, or like fibers may be utilized. For instance, a white uniform spunbond material is particularly desirable because the color exhibits good masking properties to hide menses that has passed through it. U.S. Pat. Nos. 4,801,494 to Datta, et al. and 4,908 026 to Sukiennik, et al. teach various other cover materials that can be used in the present invention.

The liner 62 can also contain a plurality of apertures (not shown) formed therethrough to permit body fluid to pass more readily into the absorbent core 66. The apertures can be randomly or uniformly arranged throughout the liner 62, or they can be located only in the narrow longitudinal band or strip arranged along the longitudinal axis X-X of the sanitary napkin 60. The apertures permit rapid penetration of body fluid down into the absorbent core 66. The size, shape, diameter any number of apertures can be varied to suit one's particular needs.

As stated above, the absorbent article also includes a baffle 64. The baffle 14 is generally liquid-impermeable and designed to face the inner surface, i.e., the crotch portion of an undergarment (not shown). The baffle 64 can permit a passage of air or vapor out of the sanitary napkin 60, while still blocking the passage of liquids. Any liquid-impermeable material can generally be utilized to form the baffle 64. For example, one suitable material that can be utilized is a microembossed polymeric film, such as polyethylene or polypropylene. In particular embodiments, a polyethylene film is utilized that has a thickness in the range of about 0.2 mils to about 5.0 mils, and particularly between about 0.5 to about 3.0 mils.

As indicated above, the sanitary napkin 60 also contains an absorbent core 66 positioned between the liner 62 and the baffle 64. In the illustrated embodiment, for example, the absorbent core 66 contains three separate and distinct absorbent members 68, 70 and 72, between any of which the compressed wetness indicator 10 may be positioned. It should be understood, however, that any number of absorbent members can be utilized in the present invention. For example, in one embodiment, only the absorbent member 72 may be utilized.

As shown, the first absorbent member 68, or intake member, is positioned between the liner 62 and the second absorbent member 70, or transfer delay member. The intake member 68 represents a significant absorbing portion of the sanitary napkin 60 and has the capability of absorbing at least about 80%, particularly about 90%, and more particularly about 95% of the body fluid deposited onto the sanitary napkin 60. In terms of amount of body fluid, the intake member 68 can absorb at least about 20 grams, particularly about 25 grams, and more particularly, about 30 or more grams of body fluid.

The intake member 68 can generally have any shape and/or size desired. For example, in one embodiment, the intake member 68 has a rectangular shape, with a length equal to or less than the overall length of the sanitary napkin 60, and a width less than the width of the sanitary napkin 60. For example, a length of between about 150 mm to about 300 mm and a width of between about 10 mm to about 40 mm can be utilized.

Typically, the intake member 68 is made of a material that is capable of rapidly transferring, in the z-direction, body fluid that is delivered to the liner 62. Because the intake member 68 is generally of a dimension narrower than the sanitary napkin 60, the sides of the intake member 68 are spaced away from the longitudinal sides of the absorbent article 60 and the body fluid is restricted to the area within the periphery of the intake member 68 before it passes down and is absorbed into the transfer delay member 70. This design enables the body fluid to be combined in the central area of the sanitary napkin 60 and to be wicked downward.

In general, any of a variety of different materials are capable of being used for the intake member 68 to accomplish the above-mentioned functions. For example, airlaid cellulosic tissues may be suitable for use in the intake member 68. The airlaid cellulosic tissue can have a basis weight ranging from about 10 grams per square meter (gsm) to about 300 gsm, and in some embodiments, between about 100 gsm to about 250 gsm. In one embodiment, the airlaid cellulosic tissue has a basis weight of about 200 gsm. The airlaid tissue can be formed from hardwood and/or softwood fibers. The airlaid tissue has a fine pore structure and provides an excellent wicking capacity, especially for menses.

A second absorbent member 70, or transfer delay member, is also positioned vertically below the intake member 68. In some embodiments, the transfer delay member 70 contains a material that is less hydrophilic than the other absorbent members, and may generally be characterized as being substantially hydrophobic. For example, the transfer delay member 70 may be a nonwoven fibrous web composed of a relatively hydrophobic material, such as polypropylene, polyethylene, polyester or the like, and also may be composed of a blend of such materials. One example of a material suitable for the transfer delay member 70 is a spunbond web composed of polypropylene, multi-lobal fibers. Further examples of suitable transfer delay member materials include spunbond webs composed of polypropylene fibers, which may be round, tri-lobal or poly-lobal in cross-sectional shape and which may be hollow or solid in structure. Typically the webs are bonded, such as by thermal bonding, over about 3% to about 30% of the web area. Other examples of suitable materials that may be used for the transfer delay member 70 are described in U.S. Pat. Nos. 4,798,603 to Meyer, et al. and 5,248,309 to Serbiak, et al., which are incorporated herein in their entirety by reference thereto for all purposes. To adjust the performance of the invention, the transfer delay member 70 may also be treated with a selected amount of surfactant to increase its initial wettability.

The transfer delay member 70 can generally have any size, such as a length of about 150 mm to about 300 mm. Typically, the length of the transfer delay member 70 is approximately equal to the length of the sanitary napkin 60. The transfer delay member 70 can also be equal in width to the intake member 68, but is typically wider. For example, the width of the transfer delay member 70 can be from between about 50 mm to about 75 mm, and particularly about 48 mm.

The transfer delay member 70 of the absorbent core 66 typically has a basis weight less than that of the other absorbent members. For example, the basis weight of the transfer delay member 20 is typically less than about 150 grams per square meter (gsm), and in some embodiments, between about 10 gsm to about 100 gsm. In one particular embodiment, the transfer delay member 70 is formed from a spunbonded web having a basis weight of about 30 gsm.

Besides the above-mentioned members, the absorbent core 66 also includes a composite member 72. For example, the composite member 72 can be a coform material. In this instance, fluids can be wicked from the transfer delay member 70 into the absorbent member 72. The composite absorbent member 72 may be formed separately from the intake member 68 and/or transfer delay member 70, or can be formed simultaneously therewith. In one embodiment, for example, the composite absorbent member 72 can be formed on the transfer delay member 70 or intake member 68, which acts a carrier during the coform process described above.

The sanitary napkin 60 may also contain other components as well. For instance, in some embodiments, the lower surface of the baffle 64 can contain an adhesive for securing the sanitary napkin 60 to an undergarment. In such instances, a backing (not shown) may be utilized to protect the adhesive side of the sanitary napkin 60 so that the adhesive remains clean prior to attachment to undergarment. The backing can generally have any desired shape or dimension. For instance, the backing can have a rectangular shape with dimension about 17 to about 21 cm in length and about 6.5 to 10.5 cm in width. The backing is designed to serve as a releasable peel strip to be removed by the user prior to attachment of the sanitary napkin 60 to the undergarment. The backing serving as a releasable peel strip can be a white Kraft paper that is coated on one side so that it can be released readily from the adhesive side of the sanitary napkin 60. The coating can be a silicone coating, such as a silicone polymer commercially available from Akrosil of Menasha, Wis.

Once formed, the sanitary napkin 60 generally functions to absorb and retain fluids, such as menses, blood, urine, and other excrements discharged by the body during a menstrual period. For example, the intake member 68 can allow the body fluid to be wicked downward in the z-direction and away from the liner 62 so that the liner 62 retains a dry and comfortable feel to the user. Moreover, the intake member 68 can also absorb a significant amount of the fluid. The transfer delay member 70 initially accepts fluid from the intake member 68 and then wicks the fluid along its length and width (-x and -y axis) before releasing the fluid to the composite absorbent member 72. The composite absorbent member 72 then wicks the fluid along its length and width (-x and -y axis) utilizing a greater extent of the absorbent capacity than the transfer delay member 70. Therefore, the composite absorbent member 72 can become completely saturated before the fluid is taken up by the transfer delay member 70. The fluid is also wicked along the length of the transfer delay member 70 and the composite absorbent member 72, thereby keeping the fluid away from the edges of the sanitary napkin 60. This allows for a greater utilization of the absorbent core 66 and helps reduce the likelihood of side leakage.

### EXAMPLES

### Example 1

A compressed wetness indicator, having a 2 cm diameter and 1 cm height, was purchased from COSCO, Cosmetic Cointissue International, Seoul Korea. The compressed wetness indicator was constructed of a nonwoven fibrous web of rayon fibers, and was shaped into a cylinder. Water was added to the compressed wetness indicator and the expansion in the z-direction (height) was recorded at the added water amount (1 milligram of water equals 0.001 milliliters water). The results are shown in Table 1:

TABLE 1

| weight of water (mg) | height (mm in z-direction) |
|---|---|
| 0 | 10 |
| 127 | 13 |
| 274 | 15 |
| 542 | 17 |
| 814 | 20 |
| 1079 | 22 |
| 1522 | 24 |
| 2019 | 25 |
| 2500 | 26 |
| 3056 | 30 |
| 3524 | 30 |
| 4067 | 32 |
| 5000 | 36 |
| 5531 | 38 |
| 6072 | 40 |
| 6542 | 40 |
| 7029 | 40 |

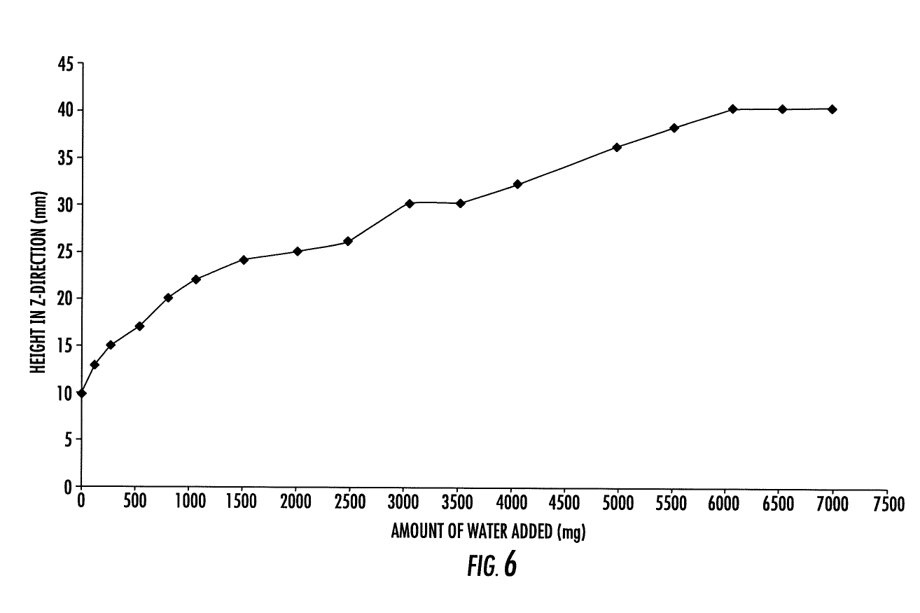

The results were also plotted into the chart shown in FIG. 6. At 1079 mg water added (1.079 mL), the diameter of the compressed wetness indicator was 2.1 cm.

### Example 2

### Physical Wetness Indicator for Diapers

A compressed wetness indicator of Example 1 was placed in the target area of a PULL-UPS diaper (Kimberly-Clark Corporation, Neenah Wis.) between the cover sheet and the absorbent core. When the diaper was wet with saline solution the compressed coin expanded in the direction towards the cover sheet (the z-direction) to a height of 4.5 cm and a diameter of 1.1 cm. This swelling would be enough to signal to the user that the diaper was wet and alert them accordingly by the pressure of the swollen object in the diaper.

### Example 3

### Physical Wetness Indicator for Feminine Pads

A compressed wetness indicator of Example 1 was sliced with a sharp knife horizontally to make a coin size disc (2 cm diameter and 3 mm thick). This sliced coin size disc was placed inside a KOTEX lightdays pad (Kimberly-Clark Corporation, Neenah Wis.) between the cover sheet and the absorbent core in the center of the pad. When the pad was wet with menses (collected from volunteer females) the compressed disc swelled up to a height of 1.5 cm while expanding to a diameter of 2.05 cm. This expansion would be sufficient to alert the user, as a physical indicator, that the pad was wet and was reaching capacity and the chance of leakage possible. The swollen lump would also serve to improve the wicking of the menstral fluid away from the body by moving closer to the body thereby improving the contract and therefore removal of the liquid.

### Example 4

### Zoned Absorbent in Feminine Pads

A compressed wetness indicator of Example 1 was sliced horizontally to make 3 mm thick and 2 cm diameter discs. Next, the discs were cut vertically to make 1 cm wide strips of 3 mm thick shapes. These were placed as a line down the center, length-wise of a KOTEX lightdays pad. They were inserted between the cover sheet and the absorbent core. The pad was then wet with saline solution and the line of compressed towel swelled to a height of 1.5 cm where the liquid had contacted the pad. This would serve, both to alert the user that the pad was wet but also to improve the removal of the liquid away from the body.

These and other modifications and variations to the present invention may be practiced by those of ordinary skill in the art, without departing from the spirit and scope of the present invention, which is more particularly set forth in the appended claims. In addition, it should be understood the aspects of the various embodiments may be interchanged both in whole or in part. Furthermore, those of ordinary skill in the art will appreciate that the foregoing description is by way of example only, and is not intended to limit the invention so further described in the appended claims.

### Example 5

### Other Nonwoven Webs Compressed

A series of nonwoven webs were made into compressed wetness indicators. Samples of each of the following fabrics were cut into 24 cm×26 cm sheets. Then, each sample was rolled and compressed, as described above, into 1 cm×2 cm compressed wetness indicators with a 20 ton pressure unit. The nonwoven webs used were 1 osy polypropylene spunbond web, 2 osy polypropylene surge layer, 3 osy coform web (70:30 polypropylene: cellulose), Wypall shop towels (Hydroknit®, Kimberly-Clark Professional, Roswell Ga.), Scott® paper towels (Kimberly-Clark, Inc, Neehan, Wis., which are described by the following U.S. Pat. Nos. 5,672,248; 5,399,412 and 5,048,589, each of which are incorporated by reference herein). When wet with water each of the pills rapidly expanded in the z-direction to a height of 4.5 cm-5 cm in under 5 seconds, while only expanding to a maximum of 2.1 cm in diameter.

### Example 6

### Z-direction Lifting Capacity of the Compressed Wetness Indicators

In order to investigate the potential z-directional lifting power of the compressed wetness indicators, a series of experiments were conducted to measure the height versus weight that these compressed pills could lift. Three compressed wetness indicators were made from spunlace webs (COSCO international, Seoul Korea) were placed in a shallow tray to form a triangle, such that each indicator was approximately 7 cm apart from each other. Next a beaker or bucket was place on top of the three pills in a manner that the pills would balance the container evenly and horizontally. The height of the container bottom from the bottom of the tray was measured. Next a known weight of water was added to the container and the total weight (container plus water) noted. Next 200 ml of water was poured onto the tray containing the pills and allowed to stand for 2 minutes. The height the compressed pills had raised the container was measured. A series of these experiments was conducted with fresh compressed wetness indicators increasing the weight of water and then height of the container raised by the pills recorded (minus the starting height). Table 2 shows the results of the study.

TABLE 2

| Weight of Water (kg) | Change in Height (cm) |
|---|---|
| 0.5 | 3.4 |
| 1.0 | 3.4 |
| 1.5 | 3.2 |
| 6.0 | 2.2 |
| 10.0 | 1.0 |
| 17.0 | 0.0 |

From these experiments, it was discovered that the compressed wetness indicators have a large pneumatic force being exerted in the z-direction upon wetting. A 2 cm diameter indicator has the surface area of 3.14 cm², therefore three indicators have 9.42 cm² (1.4 in²) surface area. These three indicators lifted a 10 kg (221 lb) weight, thus the force exerted is 15.7 psi (pounds per square inch). By comparison, a similar weight (324 mg) of superabsorbent, when wet with water, could not perform the same task as the compressed wetness indicator and, in fact, could not even vertically lift 100 g weight when wet.

What is claimed:

1. An absorbent article configured to alert a wearer of a liquid insult, the absorbent article comprising:
a liquid-permeable layer;
a liquid-impermeable layer;
an absorbent core positioned between the liquid-permeable layer and the liquid-impermeable layer; and
a compressed wetness indicator positioned between the liquid-permeable layer and the liquid-impermeable layer, wherein the compressed wetness indicator defines a x-direction, a y-direction, and a z-direction, wherein the compressed wetness indicator comprises a compression molded web and is configured to expand in the z-direction upon contact with a liquid without substantially expanding in either the x-direction or the y-direction.

2. An absorbent article as in claim 1, wherein the compressed wetness indicator is positioned between the absorbent core and the liquid-permeable layer.

3. An absorbent article as in claim 1, wherein the compressed wetness indicator is configured to at least double in size in the z-direction upon contact with a liquid.

4. An absorbent article as in claim 1, wherein the compressed wetness indicator is configured to at least triple in size in the z-direction upon contact with a liquid.

5. An absorbent article as in claim 1, wherein the compressed wetness indicator is configured to expand from about 5 times to about 10 times of its size in the z-direction upon contact with a liquid.

6. An absorbent article as in claim 1, wherein the compressed wetness indicator is configured to expand only up to about 110% of its original size in both the x-direction and the y-direction.

7. An absorbent article as in claim 1, wherein the compressed wetness indicator is configured to expand only from about 100.5% to about 105% of its original size in both the x-direction and the y-direction.

8. An absorbent article as in claim 1, wherein the compression molded web comprises a nonwoven web of pulp staple fibers.

9. An absorbent article as in claim 1, wherein the compression molded web comprises fibers formed from a synthetic polymer.

10. An absorbent article as in claim 1, wherein the compression molded web comprises a physiological cooling agent.

11. An absorbent article as in claim 1, wherein the compressed wetness indicator has a cylindrical shape.

12. An absorbent article configured to alert a wearer of a liquid insult, the absorbent article comprising:
a liquid-permeable layer;
a liquid-impermeable layer;
an absorbent core positioned between the liquid-permeable layer and the liquid-impermeable layer;
a compressed wetness indicator positioned between the liquid-permeable layer and the liquid-impermeable layer, wherein the compressed wetness indicator defines an original length in an x-direction and an y-direction, and an original height in a z-direction, wherein the compressed wetness indicator comprises a compression molded web and has an expansion ratio of greater than about 2:1.1; and
a physiological cooling agent applied to the compressed wetness indicator, wherein the physiological cooling agent is configured to provide a cooling sensation upon contact with the wearer.

13. An absorbent article as in claim 12, wherein the physiological cooling agent comprises a polyol.

14. An absorbent article as in claim 13, wherein the polyol has a chemical formula H(HCOH)ₙ₊₁H, where n is an integer from 0 to 10.

15. An absorbent article as in claim 12, wherein the compressed wetness indicator is configured to expand at least about 300% its original height in the z-direction upon contact with a liquid.

16. An absorbent article as in claim 12, wherein the compressed wetness indicator is configured to expand from about 5 times to about 10 times of its original height in the z-direction upon contact with a liquid.

17. An absorbent article as in claim 12, wherein the compressed wetness indicator is configured to expand only from about 100.5% to about 105% of its original length in both the x-direction and the y-direction.

18. A method of alerting a wearer of an absorbent article that an insult of the absorbent article has occurred, the method comprising
placing the absorbent article in contact with the wearer, wherein the absorbent article comprises a liquid-permeable layer, a liquid-impermeable layer, an absorbent core positioned between the liquid-permeable layer and the liquid-impermeable layer, and a compressed wetness indicator positioned between the liquid-permeable layer and the liquid-impermeable layer, wherein the compressed wetness indicator defines an original length in an x-direction and an y-direction, and an original height in a z-direction, wherein the compressed wetness indicator comprises a compression molded web;
wetting the compressed wetness indicator in the absorbent article with a bodily fluid, wherein upon wetting, the compressed wetness indicator expands 1-dimensionally in a direction toward the wearer, wherein the compressed wetness indicator expands according to an expansion ratio of greater than about 2:1.1.

19. A method as in claim 18, wherein the compressed wetness indicator expands according to an expansion ratio of greater than about 3:1.05.

20. A method as in claim 18, wherein the compressed wetness indicator expands according to an expansion ratio of from about 5:1.05 to about 10:1.05.
